(19) United States
(12) Patent Application Publication
(10) Pub. No.: US 2025/0143969 A1
(43) Pub. Date: May 8, 2025

(54) DEVICES, SYSTEMS, AND METHODS FOR HANGING FLUID RESERVOIRS IN SYSTEMS TO SUPPLY FLUIDS TO AN ENDOSCOPE

(71) Applicant: Boston Scientific Scimed, Inc., Maple Grove, MN (US)

(72) Inventors: Scott Edward Corbeil, Litchfield, NH (US); Rosangel Aimee Ramos Espinoza, Allston, MA (US); Ryan V. Wales, Northborough, MA (US); Kurt Nicholas Robakiewicz, Upton, MA (US); Juan M. Alvarez Azpeitia, Framingham, MA (US)

(73) Assignee: Boston Scientific Scimed, Inc., Maple Grove, MN (US)

(21) Appl. No.: 18/940,069

(22) Filed: Nov. 7, 2024

Related U.S. Application Data
(60) Provisional application No. 63/597,153, filed on Nov. 8, 2023.

(51) Int. Cl. A61J 1/16; A61B 1/015; A61J 1/10

(52) U.S. Cl. CPC A61J 1/16; A61B 1/015; A61J 1/10

(57) ABSTRACT

Hangers and hanging systems for hanging a fluid reservoir relative to an endoscope cart. An illustrative hanger may comprise a generally planar base plate and an arched member extending from an upper surface of the base plate. The arched member may include a first generally “U” shaped region and a second generally “U” shaped region. At least one arm may extend from the arched member, the at least one arm configured to receive a tubing. The second generally “U” shaped region may be configured to receive a portion of a fluid reservoir.

### CROSS-REFERENCE TO RELATED APPLICATIONS

This application claims the benefit of U.S. Provisional Patent Application Ser. No. 63/597,153 filed on Nov. 8, 2023, the disclosure of which is incorporated herein by reference.

### FIELD

This disclosure relates generally to medical fluid containers and methods, and particularly to a container and tube sets to supply fluid and/or gas to an endoscope.

### BACKGROUND

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. For example, sterile water may be used to irrigate the working lumen and/or clear organic matter (e.g., clots, fecal matter, or other masses) from the area of interest during the procedure. This may be achieved by spraying a low velocity jet of water from an orifice at the tip of the endoscope. Further, during endoscopic procedures, the video lens at the distal end of the endoscope, which is used to navigate and visualize target tissues, may be prone to becoming fouled with blood, mucous, and other debris during the procedure. Physicians need to be able to clear the lens and working lumen throughout the procedure. To generate lens wash, a connector is coupled to an endoscope umbilical via a tube set. The tube set may translate air from the endoscope umbilical to the water container or pressure vessel. A water pickup tube within the container or pressure vessel is fluid contact with the water and coupled to the connector. This allows for the pressure building in the water bottle or pressure vessel to translate the water up the water tube to the distal tip of the endoscope to clean the endoscope lens.

Endoscope fluid delivery for both lens wash and irrigation processes may include a flexible tube set with a threaded cap which connects to a one-liter (L) semi-rigid bottle of sterile water for irrigation. Commercially available tube sets may be available as either traditional two bottle tube sets or hybrid tubing sets. Traditional tubing sets consist of two separate tube sets which connect to two individual bottles of sterile water. One tube set connects to a one-liter bottle of sterile water while a second tube set is connected to second bottle for irrigation. Hybrid tubing sets, in comparison, include of a single tube set which connects to a single one-liter bottle water source, and this single system enables both irrigation and lens wash functions.

In some cases, it may be desirable to leverage off-the-shelf solution bags of sterile water or normal saline instead of bottles. There may be many advantages to using solution bags over current bottle driven systems, including, but not limited to, enabling the user to tailor the volume of the fluid reservoir to their practice needs. For example, a user can use a one-, two-, or three-liter solution bag, which are readily available to them, thus reducing or eliminating the need to change one-liter bottles over and over throughout a procedural day.

Solution bags may need to be hung. However, many endoscopy suites have space limitations, and as such have no space for a separate IV pole to hang the tubing set solution bag. As such, there is a need for a hanger that will enable the solution bag to be hung from an endoscopy cart, which typically houses capital equipment and the one-liter bottles used in current commercially available tubing sets. It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first example, a hanger for hanging a fluid reservoir relative to an endoscope cart may comprise a generally planar base plate, an arched member extending from an upper surface of the base plate, the arched member including a first generally “U” shaped region and a second generally “U” shaped region, and at least one arm extending from the arched member, the at least one arm configured to receive a tubing. The second generally “U” shaped region may be configured to receive a portion of a fluid reservoir.

Alternatively or additionally to any of the examples above, in another example, the first generally “U” shaped region may extend from the upper surface of the base plate.

Alternatively or additionally to any of the examples above, in another example, the first generally “U” shaped region may have a height greater than a raised perimeter of a shelf of an endoscope cart.

Alternatively or additionally to any of the examples above, in another example, the second generally “U” shaped region may curve away from a lateral edge of the base plate.

Alternatively or additionally to any of the examples above, in another example, the base plate may have a generally pentagonal shape.

Alternatively or additionally to any of the examples above, in another example, an apex of the base plate may be rounded.

Alternatively or additionally to any of the examples above, in another example, the hanger may further comprise a gasket secured to a lower surface of the base plate.

Alternatively or additionally to any of the examples above, in another example, the least one arm may extend generally parallel to a plane of the base plate.

Alternatively or additionally to any of the examples above, in another example, the hanger may further comprise a clamp at a free end of the at least one arm.

Alternatively or additionally to any of the examples above, in another example, the hanger may be formed at least in part from ionic silver or copper.

Alternatively or additionally to any of the examples above, in another example, the hanger may be a single monolithic structure.

In another example, a hanger for hanging a fluid reservoir relative to an endoscope cart may comprise a body portion defining a central opening, a plurality of coupling mechanisms extending through a wall of the body portion, the plurality of coupling mechanisms movable between a radially extended and a radially contracted configuration, and a hook secured to the body portion.

Alternatively or additionally to any of the examples above, in another example, the hook may be secured to the body portion by one or more fastening members.

Alternatively or additionally to any of the examples above, in another example, the one or more fastening members may be circumferentially offset from the plurality of coupling mechanisms.

Alternatively or additionally to any of the examples above, in another example, when in the radially extended configuration, the plurality of coupling mechanisms may be configured to engage one or more ribs of a shelf of a cart.

In another example, a hanger for hanging a fluid reservoir relative to an endoscope cart may comprise a generally “U” shaped body portion having a first leg including first adjustment mechanism, a second leg including a second adjustment mechanism, and a third leg, the second leg extending generally orthogonal to and between the first leg and the third leg and a curved hook portion extending from the third leg. A portion of the first leg may be configured to engage a ledge of a shelf of an endoscope cart.

Alternatively or additionally to any of the examples above, in another example, the first adjustment mechanism may be configured to adjust a length of the first leg.

Alternatively or additionally to any of the examples above, in another example, the second adjustment mechanism may be configured to adjust a length of the second leg.

Alternatively or additionally to any of the examples above, in another example, a width of the first leg may increase from a first end to an intermediate location and the width of the first leg may decrease in a stair-step manner at the intermediate location.

Alternatively or additionally to any of the examples above, in another example, the first or second adjustment mechanism may comprise a ratcheted adjustment mechanism.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3 depicts another illustrative endoscope system having an alternative fluid supply system;

FIG. 4 is a schematic perspective view of an illustrative cart that may be used to hold or mount capital equipment during an endoscopic procedure;

FIG. 5 is a perspective view of an illustrative hanger for hanging a reservoir;

FIG. 6 is a perspective view of the illustrative hanger of FIG. 5 positioned on a shelf having a raised perimeter;

FIG. 7 is a schematic side view of another illustrative hanger for hanging a reservoir positioned relative to a cart with the shelf in cross-section;

FIG. 8 is a schematic side view of another illustrative hanger that may be used to hang a reservoir relative to the cart with the shelf in cross-section;

FIG. 9 is a schematic side view of another illustrative hanger that may be used to hang a reservoir relative to the cart with the shelf in cross-section;

FIG. 10 is a bottom perspective view of an illustrative cart shelf;

FIG. 11 is a perspective view of another illustrative hanger that may be used to hang a reservoir relative to the cart;

FIG. 12A is a top view of another illustrative hanger that may be used to hang a reservoir relative to the cart;

FIG. 12B is a side view of the illustrative hanger of FIG. 12A; and

FIG. 13 is a perspective view of another illustrative hanger that that may be used to hang a reservoir relative to the cart.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. For example, sterile water may be used to irrigate the working lumen during the procedure. Further, during endoscopic procedures, the video lens at the distal end of the endoscope, which is used to navigate and visualize target tissues, may be prone to becoming fouled with blood, mucous, and other debris during the procedure. To generate lens wash, a connector is coupled to an endoscope umbilical via a tube set. The tube set may translate air from the endoscope umbilical to the water container or pressure vessel. A water pickup tube within the container or pressure vessel is fluid contact with the water and coupled to the connector. This allows for the pressure building in the water bottle or pressure vessel to translate the water up the water tube to the distal tip of the endoscope to clean the endoscope lens. The tube set used for providing irrigation fluid and/or lens wash fluid may be commonly used for a period of 24 hours across multiple endoscopic procedures. However, the same endoscope is not used for multiple patients and must be switched out between procedures. When the procedure is over, the connector is disconnected from the umbilical. The water remaining within the tube set may spill on the floor upon disconnection. Additionally, the residual pressure within the connector or pressure vessel may cause a siphon vacuum to be pulled, thus spilling all of the water in the container/pressure vessel out onto the floor via the tube set. Having a means of preventing water leaking and/or siphoning can prevent the user from needing to clean water on the floor, prevent damage to capital equipment adjacent or under the tubing connector, or requiring the user to get an additional water container or refill the water container for the next procedure. Disclosed herein are devices and systems to prevent siphoning and/or leaks of water from the water bottle and/or tube set after the procedure

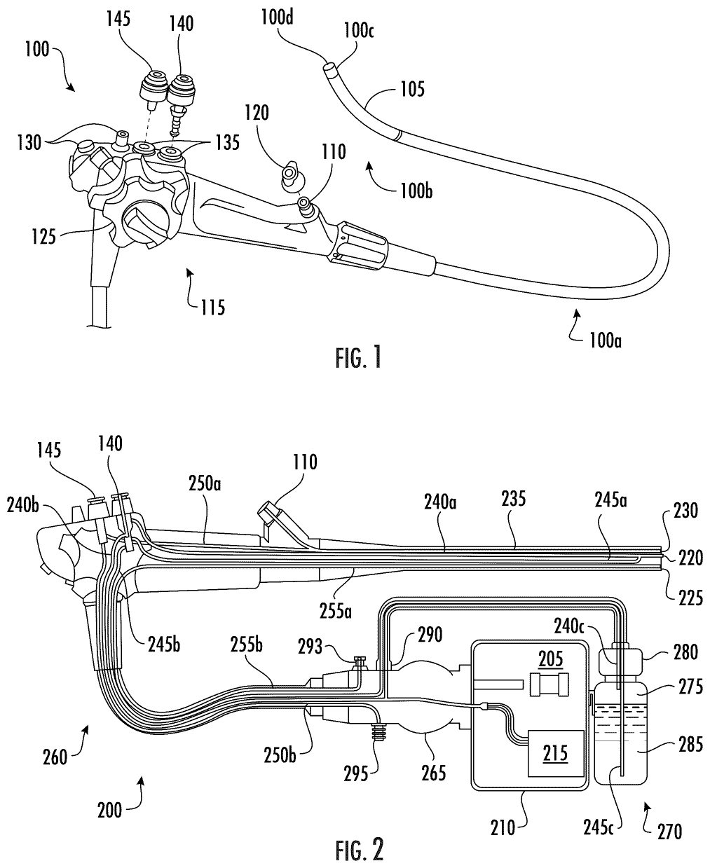

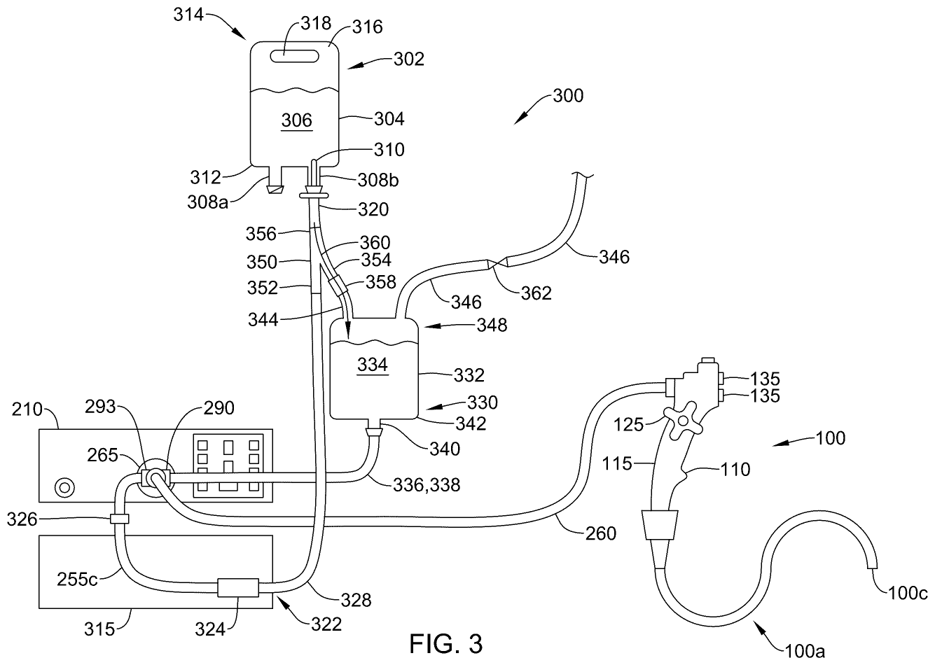

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve 140 in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connector portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top of the water reservoir. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

It is contemplated that other arrangements for the fluid sources may be used as desired. For example, in some cases, water for irrigation and lens wash may come from a same container. Some illustrative systems and method to supply fluids to the endoscope are described in commonly assigned U.S. Patent Application No. 63/419,900, titled DEVICES, SYSTEMS, AND METHODS TO SUPPLY FLUIDS TO AN ENDOSCOPE, the disclosure of which is hereby incorporated by reference.

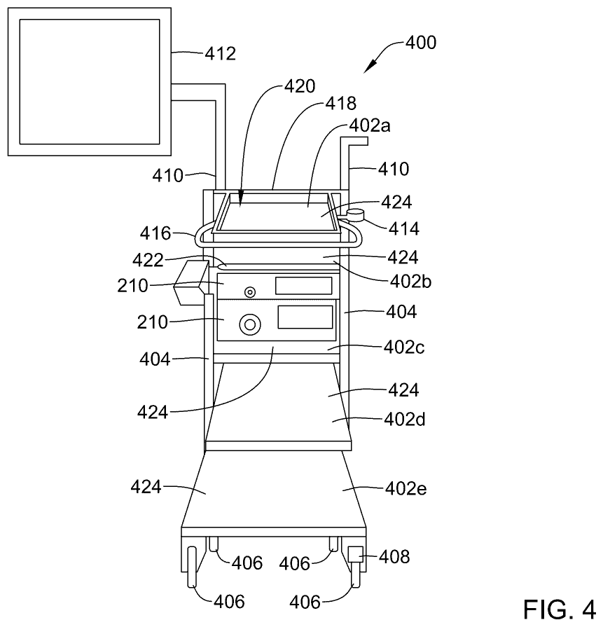

FIG. 3 depicts a schematic view of another illustrative endoscopic system 300 which may reduce the number of water reservoir changes and/or reduce opportunities for contamination during replacement of the water reservoir(s). The system 300 may include a number of advantages over the current bottle system described above. The system 300 may include components similar to the endoscope and endoscope systems described with regard to FIGS. 1-2; however, not all features may be described or shown here.

Generally, the system 300 may include a first reservoir 302 and a second reservoir 330. The first reservoir 302 may be configured to supply water or fluid for both irrigation (e.g., via the first reservoir 302) and lens wash (e.g., via the second reservoir 330). This may allow a single fluid source to be used to provide fluid for both irrigation and lens wash. While not explicitly shown, the reservoirs 302, 330 may include printed lines, numbers, or other visual indicia to allow a user to easily determine how much fluid is left in the reservoirs 302, 330.

The first reservoir 302 may include a first container 304 configured to hold a first volume of fluid 306. In the illustrated embodiment, the first container 304 is fluidly coupled to the upstream irrigation supply tubing 328 and is configured to provide fluid for irrigation to the endoscope 100. Generally, the irrigation supply tubing 328 may be a water or fluid supply line or tube for supplying water or other fluid to an endoscope. Additionally, the first container 304 may be selectively fluidly coupled to a second fluid reservoir 330. The second reservoir 330 may include a second container 332 configured to hold a second volume of fluid 334. In the illustrated embodiment, the second container 332 is fluidly coupled to the gas and lens wash supply tubing 336, 338 and is configured to provide fluid for lens wash to the endoscope 100. Generally, the lens wash supply tubing 338 may be a water or fluid supply line or tube for supplying water or other fluid to an endoscope. The gas and lens wash supply tubing 336, 338 may be coaxially arranged. For example, the gas supply tubing 336 may define a lumen that is sufficiently large in diameter to encompass a smaller diameter lens wash tubing 338, coaxially received within the gas supply tubing 336, as well as provide air to the water source in an annular space surrounding the lens wash tubing 338 to pressurize the second reservoir 330. The lens wash supply tubing 338 may be configured to exit the lumen defined by the coaxial gas supply tubing 336 in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265. In other embodiments, the gas and lens wash supply tubing 336, 338 may be arranged in a side-by-side arrangement.

The first and second containers 304, 332 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first and second containers 304, 332 may be entirely translucent, entirely opaque, or combinations thereof. In some cases, the first and second containers 304, 332 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the first and second containers 304, 332 may be variable. For example, the volume of the first container 304 and/or the second container 332 may be 500 milliliters (mL) or greater, 1000 mL or greater, 2000 mL or greater, 3000 mL or greater, 4000 mL or greater, etc. The volume may be less than 500 mL or greater than 4000 mL, as desired. One or both of the first and second reservoirs 302, 330 may be pre-filled (e.g., prior to entering the procedure suite or at the time of manufacturing) with water or other fluid. In some cases, the clinician may select the reservoir(s) 302, 330 from a plurality of differently sized available reservoirs based on the number and/or types of procedures expected for a typical day or the specific day. In the illustrated embodiments, the first reservoir 302 may supply fluid to the second reservoir 330. By selecting a first reservoir 302 having a volume large enough to accommodate an entire day of procedures, the need for replacing the sterile fluid source (e.g., the first reservoir 302) may be reduced or eliminated. In some cases, the first reservoir 302 may be used to periodically refill the second reservoir 330. Thus, the volume of the first reservoir 302 may be greater than the volume of the second reservoir 330, although this is not required. It is further contemplated that, in some embodiments, one or both of the first or second reservoirs 302, 330 may be a rigid bottle.

It is contemplated that flexible bags may utilize less plastic (or other material) than a bottle designed to hold a similar amount of fluid. Thus, the use of a flexible bag as a fluid reservoir 302, 330 may increase the level of environmental sustainability of the system 300. For example, if the user sets up the system with a 3000 mL (3 liter) bag reservoir 302 and therefore does not need to utilize three individual one-liter bottles, a significant reduction of waste may be realized. It is further contemplated that when disposed of or discarded, a flexible bag reservoir may occupy less volume than a bottle capable of holding an equivalent amount of fluid.

The first reservoir 302 may further include one or more ports 308a, 308b, such as, but not limited to a spike port or a septum port, extending from and in selective fluid communication with an interior of the first container 304. The ports 308a, 308b may be formed as a monolithic structure with the first container 304. The ports 308a, 308b may be generally tubular structures with each port 308a, 308b defining a lumen extending therethrough. The lumens of the ports 308a, 308b may be configured to selectively fluidly couple the interior of the first container 304 with another component, such as, but not limited to, a fluid or water supply tube. In some embodiments, the ports 308a, 308b may be positioned adjacent to a bottom end 312 of the first reservoir 302. However, this is not required. The ports 308a, 308b may be positioned in other locations, as desired. If the ports 308a, 308b are positioned at a location other than the bottom end 312 of the first container 304, a dip tube or tube extension may be required to access the fluid at the bottom of the first container 304. In some cases, at least one port 308b may be configured to be coupled to the upstream irrigation tubing (or water supply tube) 328 while another port 308a may be configured to allow the user to add additives to the fluid 306. In other examples, the upstream irrigation tubing 328 may be coupled to the first port 308a while the second reservoir 330 is in fluid communication with the second port 308b. While the first reservoir 302 is illustrated as including two ports 308a, 308b, the first reservoir 302 may include one port or more than two ports, as desired.

While not explicitly shown, the ports 308a, 308b may each include a removable cap or seal configured to form a fluid tight seal with the port 308a, 308b. The removable cap or seal may help to maintain the sterility of the ports 308a, 308b. The removable cap or seal may be coupled to a free end of the ports 308a, 308b using a number of different techniques. For example, the cap or seal may be coupled to the port 308a, 308b using a threaded engagement, a friction fit, a snap fit, etc. In other instances, the cap or seal may be removed through a twisting motion configured to break the cap or seal from the port 308a, 308b. Once the cap or seal has been removed, the port 308a, 308b may be pierced with a spike tip or spike port adaptor 310 that is coupled to the upstream irrigation tubing 328. For example, in addition to the removable cap or seal, the port 308a, 308b may include an internal seal disposed within a lumen of the port 308a, 308b that may be punctured or pierced by the spike port adaptor 310. The internal seal may be configured to prevent fluid 306 from leaking from the first container 304 prior to the spike port adaptor 310 being inserted into the port 308a, 308b. In some embodiments, the internal seal may be self-sealing such that upon removal of the spike port adaptor 310 fluid is prevented from leaking from the port 308a, 308b. The outer surface of the spike port adaptor 310 may form an interference fit with the inner surface of the port 308a, 308b. The fit and/or coupling between the spike port adaptor 310 and the port 308a, 308b may be sufficient to remain in place when the irrigation supply tube 328, branched connector 350, and/or other tubing sets are coupled to the spike port adaptor 310. It is contemplated that the spike port adaptor 310 may be inserted into one of the ports 308a, 308b utilizing universally used aseptic techniques such as those used with IV fluid bags. This may help reduce infection risk by maintaining sterile components, not introducing contaminants into the fluid 306, etc. It is further contemplated that additives may be added to the fluid 306 using similar aseptic techniques via one of the ports 308a, 308b.

The first reservoir 302 may include a handle 316 positioned adjacent to a top portion 314 thereof. The handle 316 may define an opening or through hole 318 for receiving a hand or hook therethrough to carry the first reservoir 302. In some cases, the handle 316 may include an undulating surface configured to provide a more ergonomic grip for the user. It is contemplated that the handle 316 may be formed from a similar material as the first container 304 or a different material, as desired. In some examples, the handle 316 may be formed from polyethylene terephthalate (PET), polypropylene (PP), etc. The handle 316 may allow the first reservoir 302 to be hung from a hook, such as, but not limited to an IV stand. Hanging the first reservoir 302 may allow the first reservoir 302 to be positioned above the level of an endoscope cart which may enable the user to see the fluid 306 level at any time. This may help the clinician avoid running out of fluid during a procedure. Additionally, elevating the reservoir may eliminate the need for the clinician to bend or stoop during setup of the system 300 and/or to change the first reservoir 302. In some cases, head pressure generated from elevating the first reservoir 302 may enable rapid priming of the irrigation circuit (and/or lens wash circuit if so connected) which may save time during setup. It is further contemplated that hanging the first reservoir 302 from a hook or IV stand may allow the first reservoir 302 to be positioned away from expensive capital equipment thus reducing or eliminating the potential for fluid running or flowing inadvertently onto the capital equipment and causing damage or destruction.

The first reservoir 302 may be connected in fluid communication with a lumen of the upstream irrigation supply tube 328. The upstream irrigation supply tube 328 extends from a second end region 322 external to the container 304 and positioned within a pump head 324 of the peristaltic irrigation pump 315 to a first end 320. The first end 320 of the upstream irrigation supply tube 328 is coupled to the spike port adaptor 310 which in turn is configured to extend through a lumen of the port 308b and pierce a seal within the lumen of the port 308b to fluidly couple the interior of the container 304 with the lumen of the upstream irrigation supply tube 328. The second end of the upstream irrigation supply tube 328 is configured to be fluidly coupled with an irrigation lumen of the endoscope 100. When irrigation water is required, fluid is pumped from the first container 304 by operating the irrigation pump 315, such as by depressing a footswitch (not shown), and flows from the first reservoir 302, through the upstream irrigation supply tubing 328 and a branched connector 350, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 326 positioned in line with the downstream irrigation supply tubing 255c. The flow control valve 326 may prevent the unintentional flow of fluid from the first container 304 to the endoscope 100. In some cases, the flow control valve 326 may be configured to open when the pressure within the downstream irrigation supply line 255c reaches a predetermined minimum pressure. It is contemplated that the predetermined minimum pressure may be greater than the head pressure created by the height differential between the first reservoir 302 and the irrigation pump 315. The flow control valve 326 may also prevent fluid from leaking from the downstream irrigation supply tube 255c when the endoscope 100 is changed between patients and the tubing set connector is separated from the endoscope water port.

In some embodiments, the irrigation pump 315 may be omitted. For example, the reservoir 302 may be inserted into a compression sleeve. When irrigation fluid is desired, the compression sleeve may be activated to exert pressure on an outer surface of the reservoir 302 and to provide the required pressure to perform irrigation at the distal end of the endoscope 100. In another embodiment, the reservoir 302 may be inserted into a compression sleeve which applies constant pressure to the reservoir 302 with a flow switch positioned along irrigation supply tubing 328 to provide binary control of irrigation flow.

The second reservoir 330 may further include one or more ports 340, such as, but not limited to a spike port or a septum port, extending from and in selective fluid communication with an interior of the second container 332. The port 340 may be formed as a monolithic structure with the second container 332. The port 340 may be a generally tubular structure with the port 340 defining a lumen extending therethrough. The lumen of the port 340 may be configured to selectively fluidly couple the interior of the second container 332 with another component, such as, but not limited to, fluid/water/gas supply tube(s). In some cases, the port 340 may be configured to be coupled to the gas and lens wash supply tubing 336, 338. In some embodiments, the port 340 may be positioned adjacent to a bottom end 342 of the second reservoir 330. However, this is not required. The port 340 may be positioned in other locations, as desired. If the port 340 is positioned at a location other than the bottom end 342 of the second container 332, a dip tube or tube extension may be required (e.g., coupled to the lens wash supply tubing 338) to access the fluid at the bottom of the second container 332. While the second reservoir 330 is illustrated as including one port 340, the second reservoir 330 may include more than one port, as desired.

While not explicitly shown, the port 340 may include a removable cap or seal configured to form a fluid tight seal with the port 340. The removable cap or seal may help to maintain the sterility of the port 340. The removable cap or seal may be coupled to a free end of the port 340 using a number of different techniques. For example, the cap or seal may be coupled to the port 340 using a threaded engagement, a friction fit, a snap fit, etc., or may be fixedly coupled using a number of techniques such as adhesive or solvent bonding. In other instances, the cap or seal may be removed through a twisting motion configured to break the cap or seal from the port 340. Once the cap or seal has been removed, the port 340 may be pierced with a spike tip or spike port adaptor (not explicitly shown) that is coupled to the gas and lens wash supply tubing 336, 338. For example, in addition to the removable cap or seal, the port 340 may include an internal seal disposed within a lumen of the port 340 that may be punctured or pierced by the spike port adaptor. The internal seal may be configured to prevent fluid 334 from leaking from the second container 332 prior to the spike port adaptor being inserted into the port 340. In some embodiments, the internal seal may be self-sealing such that upon removal of the spike port adaptor fluid is prevented from leaking from the port 340. The outer surface of the spike port adaptor may form an interference fit with the inner surface of the port 340. The fit and/or coupling between the spike port adaptor and the port 340 may be sufficient to remain in place when the gas and fluid supply tubing 336, 338 and/or other tubing sets are coupled to the spike port adaptor. It is contemplated that the spike port adaptor may be inserted into the port 340 utilizing universally used aseptic techniques such as those used with IV fluid bags. This may help reduce infection risk by maintaining sterile components, not introducing contaminants into the fluid 334, etc. It is further contemplated that additives may be added to the fluid 334 using similar aseptic techniques via the port 340, if so desired. In some cases, other coupling mechanisms may be used as desired to couple the gas and lens wash supply tubing 336, 338 to the port 340. Some illustrative coupling mechanisms may include, but are not limited to, threaded engagements, snap fits, friction fits, quick connect style couplers, etc., or may be fixedly coupled using a number of techniques such as adhesive or solvent bonding.

The gas supply tubing 336 extends from a second end external to the second container 332 to the port 340. The gas supply tubing 336 may extend into the interior of the second container 332 and terminate within a reservoir gap (e.g., above the level of the fluid 334). However, in some cases, the gas supply tubing 336 may terminate within the fluid 334. A lumen extends through the gas supply tubing 336 for receiving a flow of air and/or gas therethrough. The lumen of the gas supply tubing 336 may be in operative fluid communication with a top portion of the interior of the second container 332. The lens wash supply tubing 338 extends from a second end external to the second reservoir 330 to a first end in fluid communication with a bottom portion 342 of the second container 332. In some embodiments, the lens wash supply tubing 338 may terminate at the port 340. A lumen extends through the lens wash supply tubing 338 for receiving a flow of fluid therethrough. The lumen of the lens wash supply 338 is in selective operative fluid communication with a bottom portion 342 of the second container 332. In the illustrated embodiment, the gas supply tubing 336 and the lens wash supply tubing 338 may couple to the second container 332 through a single or common opening (e.g., port 340). For example, the gas supply tubing 336 and the lens wash supply tubing 338 may be coaxially arranged. However, this is not required. In some cases, the gas supply tubing 336 and the lens wash supply tubing 338 may extend in a side by side arrangement or may be separately connected to the second container 332 in different locations.

The second container 332 may further include a first fluid inlet 344 and a second fluid inlet 346. While the first and second fluid inlets 344, 346 are illustrated as being adjacent to or extending from a top portion 348 of the second container 332, the first and/or second fluid inlets 344, 346 may be positioned at other locations about the second container 332, as desired. In some embodiments, the first and/or second fluid inlets 344, 346 may be tubular members formed as a single monolithic structure with the second container 332. In other embodiments, the first and/or second fluid inlets 344, 346 may include tubular components releasably coupled to ports (similar in form and function to port 340) formed in or with the container 332.

The first fluid inlet 344 may be in selective fluid communication with the first reservoir 302. For example, a branched connector 350 may be positioned in-line with the upstream irrigation tubing 328. In some embodiments, the branched connector 350 may be a “Y” connector or a “T” connector having an inlet leg 356 defining a first fluid inlet, a first outlet leg 352 defining a first fluid outlet, and a second outlet leg 354 defining a second fluid outlet. However, it is contemplated that the branched connector 350 may include more than one fluid inlet and fewer than two or more than two fluid outlets, if so desired.

The branched connector 350 may be positioned in-line with the upstream irrigation tubing 328 such that the inlet leg 356 and the first outlet leg 352 are fluidly coupled with the lumen of the upstream irrigation tubing 328. Fluid may flow from the first reservoir 302, through the upstream irrigation tubing 328, through the branched connector 350 and again through the upstream irrigation tubing 328. The branched connector 350 may be positioned such that the inlet leg 356 is upstream of the outlet legs 352, 354 relative to a flow of irrigation fluid. In some embodiments, the branched connector 350 and the spike port adaptor 310 may be molded or formed as a single monolithic structure. It is contemplated that this may reduce connection points in the fluid circuit. In such an instance, the first end 320 of the irrigation supply tubing 328 may be fluidly coupled to the first outlet leg 352 of the branched connector 350.

The second outlet leg 354 may be fluidly coupled to the first fluid inlet 344 of the second reservoir 330. A flow control mechanism, such as, but not limited to, a one-way valve 358 may be positioned between the second fluid outlet of the second outlet leg 354 and the first fluid inlet 344 of the second reservoir 330 to selectively fluidly couple the second container 332 with the first container 304. The one-way valve 358 may be configured to be opened to allow fluid to selectively pass from the first reservoir 302 to the second reservoir 330 while preventing fluid (e.g., gas, water, or other fluid) from exiting the second container 332 and entering the irrigation supply tubing 328 and/or the first container 304. In some embodiments, the one-way valve 358 may be replaced with a clamp which may compress the first fluid inlet 344 to selectively fluidly isolate the second container 332 from the first container 304 and removed to selectively fluidly couple the second container 332 with the first container 304. In yet other embodiments, the one-way valve 358 may be replaced with a spring-loaded valve, a stopcock, or other two-way valve. When it is desired to add fluid to the second reservoir 330 from the first reservoir 302, the one-way valve 358 (or other flow control mechanism) may be opened or released. Fluid may then be at least partially diverted from the irrigation supply tubing 328 through the second outlet leg 354 of the branched connector 350 and into the second container 332 along flow path 360. Fluid may be added to the second container 332 while the irrigation pump 315 is running or while the irrigation pump 315 is idle, as desired.

The second fluid inlet (or gas supply tube) 346 of the second container 332 may be an alternative gas supply tubing configured to be coupled to an alternative gas supply (e.g., CO₂ hospital house gas source). The second fluid inlet 346 may extend from a second end external to the second container 332 to a first end coupled to the second container 332. The alternative gas supply may be used to pressurize the second container 332 to supply lens wash to the endoscope 100 and/or to provide insufflation. A lumen extends through the second fluid inlet 346 for receiving a flow of gas therethrough. The lumen of the second fluid inlet 346 is in operative fluid communication with a top portion of the second container 332. The flow of the CO₂ through the system 300 may be similar to that described above. For example, in the neutral state, CO₂ gas flows through the second fluid inlet 346 into the second container 332, up the gas supply tubing 336 to the connector portion 265, up the gas feed line 240b in the umbilical 260, and is vented through the gas/water valve 140 to atmosphere. In the first position, the user closes off the vent hole in the gas/water valve 140, and the CO₂ gas is flowed through the second fluid inlet 346 into the second container 332, up the gas supply tubing 336 to the connector portion 265, through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the second reservoir 330 is maintained by delivering gas through the second fluid inlet 346. It is contemplated that the one-way valve 358 is in the closed configuration during delivery of the CO₂ gas to allow the container 332 to pressurize. In some instances, the one-way valve 358 may be configured to close without user intervention in response to the delivery of CO₂ to the second container 332. In some embodiments, the system 300 may include a branched connector (such as, but not limited to a “Y” or “T” connector) at the second fluid inlet 346 to allow either air or CO₂ to be used for pressurization or insufflation. It is further contemplated that the second fluid inlet 346 may include a pressure relief valve 362, such as, but not limited to, a 3-way stopcock, a clamp, or a spring-loaded valve, to vent pressure within the second container 332 and/or to block a flow of pressurized gas to the second container 332 during refilling of the second container 332, during procedure change-overs, and/or during equipment change-overs.

It is contemplated that the use of a flexible bag in place of a rigid bottle for the second reservoir 330 may reduce or eliminate the risk of air leaking from bottle and cap connections. This may eliminate the need for clinicians to attempt to remedy the leak by adjusting the cap and bottle assemblies or from discarding a cap and/or bottle if the leak cannot be remedied.

As the pressurized second container 332 is fluidly isolated from the first container 304 when the one-way valve 358 is closed, it is contemplated that the clinician may replace the first reservoir 302 with a new (full) reservoir without losing patient insufflation. Loss of patient insufflation may result in a loss of position of the endoscope 100 within the body. In current one or two bottle systems, it may not be possible to replace the water reservoirs without loss of patient insufflation.

If there is a need to replace the first reservoir 302 with a new full bag, for example when the first reservoir 302 is empty or near empty, the user may hang the new bag near the first reservoir 302 to be replaced. The user may then disengage the spike port adaptor 310 from the port 308b and insert the spike port adaptor 310 into a port of the new bag. This may be performed without requiring the clinician to bend or stoop to access the first reservoir 302. The port 308b may self-seal to prevent fluid leaks from the first reservoir 302 being replaced. This method of replacing the first reservoir 302 may have a lower risk of introducing contaminants into the systems relative to traditional bottle systems. For example, the change out method described herein may allow the first reservoir 302 to be changed out without having tubing dangling from a cap (as in a bottle system). Further, the system 300 may remain largely closed as the first reservoir 302 is changed out.

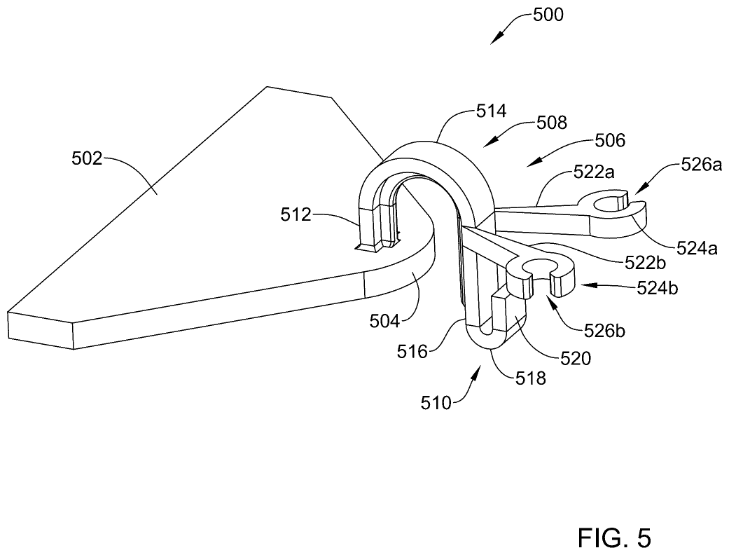

FIG. 4 is a schematic perspective view of an illustrative cart 400 that may be used to hold or mount capital equipment during an endoscopic procedure. The illustrative cart 400 may include a plurality of shelves 402a-e movably or fixedly secured to a frame 404. The cart 400 may include any number of shelves 402a-e desired. The shelves 402a-e may be configured to store equipment thereon. For example, one or more processing units 210 may be positioned one or more of the shelves 402a-e. The shelves 402a-e may include differing structures to accommodate different components of the endoscope system 200. For example, some shelves 402c-e may be generally planar to allow equipment to be easily positioned thereon and/or removed therefrom. Other shelves 402a may include a raised perimeter 418 defining an inner cavity or recess 420. This may help prevent items from falling off the shelf 402a-e. In yet other examples, shelves 402b may include a raised lip 422 along one or more edges thereof to facilitate gripping of the shelf 402b. Each shelf 402a-e may include an upper surface 424 upon which equipment may be positioned.

The cart 400 may further include a plurality of wheels 406 to facilitate positioning of the cart 400 within the procedure suite. In some cases, one or more of the wheels 406 may include a locking mechanism 408 configured to prevent movement of the wheel 406 and/or cart 400. One or more arms or poles 410 may extend from the frame 404. The poles 410 may be configured to mount various components thereto, such as, but not limited to, a display screen 412. In some cases, the poles 410 may include features configured to receive tube sets, endoscope handles, and the like. Alternatively, or additionally, mounting features 414 for receiving tube sets, the endoscope handle, and the like may be mounted to one or more of the shelves 402a-e and/or the frame 404. The cart 400 may include one or more handle 416. The handles 416 may be formed as a single monolithic structure with or may be coupled to one of the shelves 402a-e. Alternatively, or additionally, the handles 416 may be formed as a single monolithic structure with or may be coupled to the frame 404. It should be understood that the cart 400 may include additional features not expressly described herein. Further the shelves 402a-e, frame 404, poles 410, and/or other features may be arranged in any manner desired.

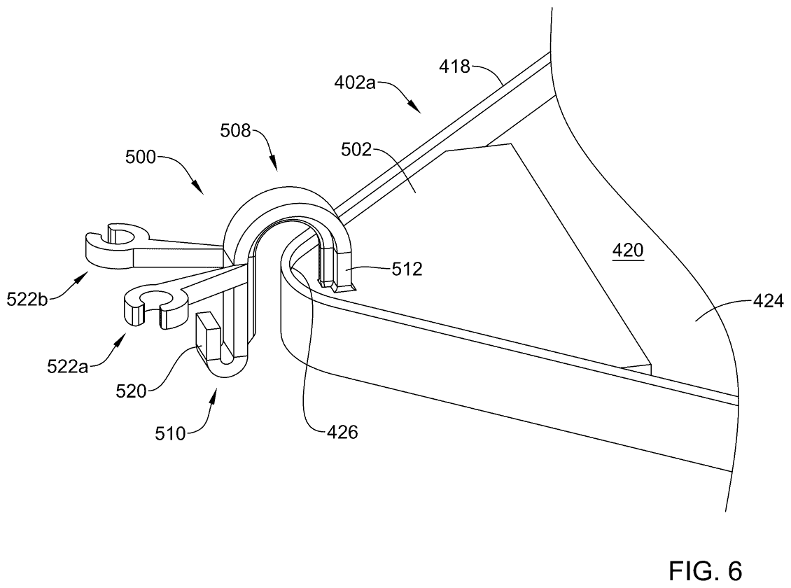

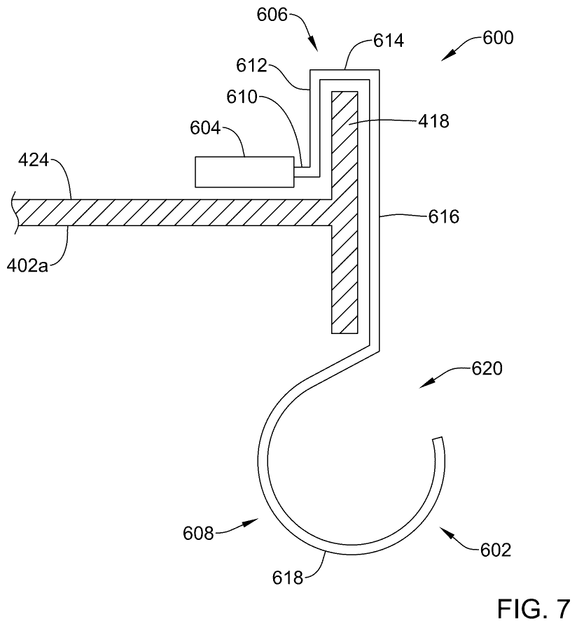

As described herein, it may be desirable to mount one or more of the reservoirs 302, 330 from the cart 400. FIG. 5 is a perspective view of an illustrative hanger 500 for hanging a reservoir 302, 330 relative to the cart 400. FIG. 6 is a perspective view of the illustrative hanger 500 positioned on a shelf 402a having a raised perimeter 418. The hanger may include a generally planar base plate 502. The base plate 502 may have a generally pentagonal shape. In some cases, a vertex 504 of the base plate 502 may be rounded to conform to an inner corner 426 of the shelf 402a-e. However, the vertex 504 may be take other shapes as desired. For example, the vertex 504 may take the form of a traditional vertex. The angle of intersection of the sides of the base plate 502 and/or the length of the sides of the base plate 502 may be varied to fit within the corner 426 of the shelf 402a. The base plate 502 may take other shapes, such as, but not limited to, triangular, square, rectangular, polygonal, eccentric shapes, oblong, and the like. Generally, a lower surface of the base plate 502 may be configured to rest on a surface 424 of the shelf 402a at a corner 426 of the shelf 402a. However, the base plate 502 may be positioned at other locations along the perimeter of the shelf 402a, if so desired. It is further contemplated that that hanger 500 may be used with shelves 402b-e free from a raised perimeter 418. The base plate 502 may be fixedly or releasably secured to the shelf 402a. For example, the base plate 502 may be secured to the shelf 402a using adhesive, adhesive tape, screws, bolts, snap locks, anchors, hook and loop strips, or the like.

In some embodiments, a gasket (not explicitly shown) may be provided or secured around a perimeter of the base plate 502 and/or to a lower surface configured to be in contact with the surface 424 of the shelf 402a. The gasket may be formed from a deformable material configured to form a seal between the base plate 502 and the surface 424 of the shelf 402a and/or the raised perimeter 418 thereof. The gasket may prevent debris and/or potentially harmful micro-organisms from being trapped and propagating between the base plate 502 and the shelf 402a.

An arched member 506 may extend from a first or upper surface of the base plate 502. The arched member 506 may extend from an upper surface of the base plate 502 opposite the lower surface which rests on the surface 424 of the shelf 402a. The arched member 506 may include a first generally “U” or horseshoe shaped region 508 and a second generally “U” or horseshoe shaped region 510. The first generally “U” shaped region 508 may curve in a first direction and the second generally “U” shaped region may curve in a second direction opposite the first direction. In an illustrative example, the open channel of the first “U” shaped region 508 may open in a first direction while the open channel of the second “U” shaped region 510 may open in a second direction opposite the first direction. It is contemplated that the other geometric connections may be used, as desired. The first region 508 may be configured to extend over and laterally beyond the raised perimeter 418 of the shelf 402a while the second region 510 may be configured to receive a handle 316 (or other hanging means) of the reservoir 302, 330. The first region 508 may include a first leg 512 extending generally orthogonal to a plane of the base plate 502. A first curved bridge portion 514 may connect the first leg 512 with a second leg 516. The second leg 516 may be laterally spaced from and extend generally parallel to the first leg 512. A second curved bridge portion 518 may connect the second leg 516 with a third leg 520. The third leg 520 may be laterally spaced from and extend generally parallel to the second leg 516. The second leg 516 may form a portion of the first region 508 and a portion of the second region 510.

One or more arms 522a, 522b may extend laterally from the second leg 516. In some cases, the one or more arms 522a-b may extend generally parallel to the plane of the base plate 502. The one or more arms 522a-b may each include a clamp 524a, 524b at a free end thereof. The clamps 524a-b may define a lateral opening 526a, 526b configured to receive a tube set therethrough. A central opening 528a, 528b may extend through a thickness of the clamps 524a-b. In some cases, the clamps 524a-b may have a generally “C” shape. The clamps 524a-b may be configured to receive an endoscope end of the irrigation supply tubing 328 or an endoscope end of the gas and lens wash supply tubing 336, 338 during equipment change over. It is further contemplated that the clamps 524a-b may be configured to receive the fluid supply ends of the irrigation supply tubing 328 or the gas and lens wash supply tubing 336, 338 during a reservoir 302, 330 change out.

In some embodiments, the hanger 500 may be formed as a single monolithic structure. In other embodiments, the hanger 500 may be formed from two or more components that are subsequently coupled together. The hanger 500 may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like. It is further contemplated that the hanger 500 may be formed from a material which is chemically resistant to allow the user to use bacteriostatic and bactericidal cleaners on and around the hanger 500. In some examples, the hanger 500 may be formed, at least in part, with materials having bactericidal properties, such as, but not limited to, ionic-silver, copper or other chemical species or compounds known to inhibit microorganism growth.

To hang the reservoir 302 from the hanger 500, the hanger 500 may first be secured to the desired shelf 402a-e at the desired location. Next, the handle 316 (or other hanging means) of the reservoir 302 may be positioned over the third leg 520 of the arched member 506. The handle 316 may rest within the second bridge portion 518 to hang the reservoir 302 from the hanger 500.

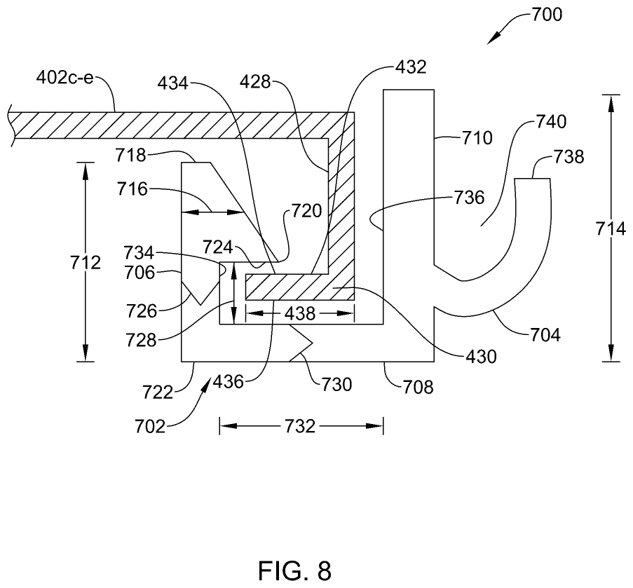

FIG. 7 is a schematic side view of another illustrative hanger 600 for hanging a reservoir positioned relative to a shelf 402a of a cart 400, with the shelf 402a in cross-section. The hanger 600 may include a hook 602 configured to hang over the raised perimeter 418 of the shelf 402a. The hook 602 may have a serpentine shape that is configured to accommodate the raised perimeter 418 and distribute the load under the shelf 402a. The hanger 600 may include a weighted body portion 604 configured to rest on a surface 424 of the shelf 402a. The weighted body portion 604 have a weight sufficient to act as a counterweight for up to a three-liter reservoir. The weighted body portion 604 may take any shaped desired, such as, but not limited to, cylindrical, cubic, rectangular prism, triangular prism, and the like.

The hook 602 may be coupled to or formed as single monolithic structure with the weighted body portion 604. The hook 602 may include a first end region 606 extending from the weighted body portion 604 and a second end region 608 configured to hang below the shelf 402a. The first end region 606 may be configured to accommodate the raised perimeter 418 of the shelf 402a. For example, the first end region 606 may include a first segment 610 extending from the weighted body portion 604 and generally parallel to the plane of the surface 424 of the shelf 402a. A second segment 612 may extend from and generally orthogonal to the first segment 610. The second segment 612 may extend in a direction away from the surface 424 of the shelf 402a. The second segment 612 may have a height that is at least as great as the raised perimeter, or greater. A third segment 614 may extend from and generally orthogonal to the second segment 612. The third segment 614 may extend in a direction away from an interior of the shelf 402a. A fourth segment 616 may extend from and generally orthogonal to the third segment 614. The fourth segment 616 may extend in a direction towards a lower surface of the shelf 402a. Collectively, the second, third, and fourth segments 612, 614, 616 may have a generally “U” or horseshoe shape that is configured to extend up and over the raised perimeter 418 and back down. The configuration of the second, third, and fourth segments 612, 614, 616 may be varied to accommodate the structure of the shelf 402a-e. In some examples, the second and third segments 612, 614 may be omitted. For example, when the shelf is generally planar, the first segment 610 may couple directly with the fourth segment 616. In some embodiments, the segments 610, 612, 614, 616 of the first end region 606 may be fixedly or movably coupled to one another or formed as a single monolithic structure. In other embodiments, the segments 610, 612, 614, 616 of the first end region 606 may be movably (e.g., pivotably) coupled to one another to form different geometries.

The second region 608 may be a curved segment 618 extending from an end of the fourth segment 616 of the first region 606. The second end region 608 may be fixedly or movably coupled to the first region 606 or may be formed as a single monolithic structure with the first region 606. In other embodiments, the second end region 608 may be movably (e.g., pivotably) coupled to the first region 606 to form different geometries. The curved segment 618 may extend laterally inwards before curving back on itself and extending laterally outwards and upwards relative to an outer surface of the shelf 402a. The curved segment 618 may form less than a 360° arc. For example, the curved segment 618 may include an opening 620 for allowing the handle 316 of a reservoir 302 to pass therethrough. The handle 316 of the reservoir 302 may rest on an inner surface of the curved segment 618 to support the reservoir 302.

In some embodiments, the hanger 600 may be formed as a single monolithic structure. In other embodiments, the hanger 600 may be formed from two or more components that are subsequently coupled together. The hanger 600 may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like. It is further contemplated that the hanger 600 may be formed from a material which is chemically resistant to allow the user to use bacteriostatic and bactericidal cleaners on and around the hanger 600. In some examples, the hanger 600 may be formed, at least in part, with materials having bactericidal properties, such as, but not limited to, ionic-silver, copper or other chemical species or compounds known to inhibit microorganism growth.

The hanger 600 may be positioned at any location along the perimeter of the shelf 402a, as desired. As the load of the reservoir 302 may be transferred through the weighted body portion 604 to the surface 424 of the shelf 402a, the hanger 600 may not be secured to the shelf 402a. However, the body portion 604 may be fixedly or releasably secured to the shelf 402a. For example, the body portion 604 may be secured to the shelf 402a using adhesive, adhesive tape, screws, bolts, snap locks, anchors, hook and loop strips, or the like.

In some embodiments, a gasket (not explicitly shown) may be provided around a perimeter of the body portion 604, along a surface configured to be in contact with the surface 424 of the shelf 402a, and/or along the hook 602. The gasket may be made from a deformable material configured to form a seal between the body portion 604 and the surface 424 of the shelf 402a and/or the raised perimeter 418 thereof. The gasket may prevent debris and/or potentially harmful micro-organisms from being trapped and propagating between the body portion 604 and the shelf 402a.

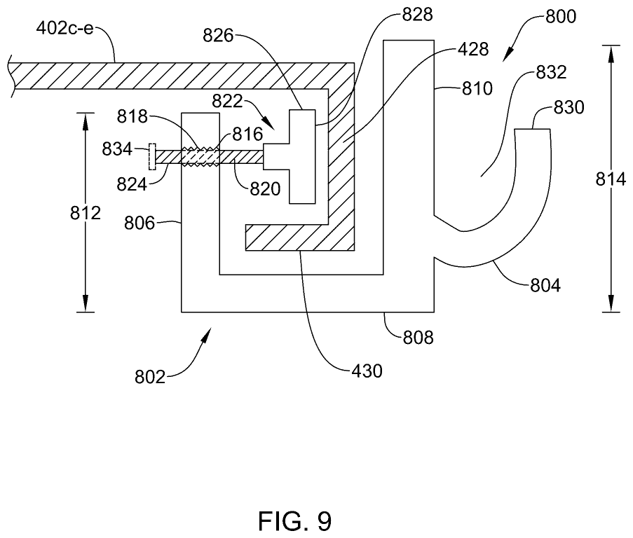

FIG. 8 is a schematic side view of another illustrative hanger 700 that may be used to hang a reservoir 302 relative to the cart 400 with the shelf 402c-e in cross-section. The cart shelf 402c-e may have a generally planar upper surface, although this is not required. The illustrative hanger 700 may be used with shelves having a raised perimeter or upwardly extending lip, as desired. The cart shelf 402c-e may include a downwardly extending (e.g., towards the wheels 406) portion or perimeter edge 428 and an inwardly extending portion 430. The inwardly extending portion 430 may extend towards a center of the shelf 402c-e and may extend generally parallel to the main portion of the shelf 402c-e. The downwardly extending portion or perimeter edge 428 may extend generally orthogonal to and between the main portion of the shelf 402c-e and the inwardly extending portion 430. The inwardly extending portion 430 may have a length and/or width 438 that is less than length and/or width of the shelf 402c-e to define a rim or ledge 432 extending about a perimeter of the shelf 402c-e below a surface 424 of the shelf 402c-e.

The hanger 700 may be configured to clamp to the perimeter edge 428 and/or the ledge 432 of the shelf 402c-e. The hanger 700 may include a generally “U” shaped body portion 702 configured to be releasably secured to the shelf 402c-e and a curved hook portion 704 configured to receive the handle 316 of the reservoir 302. The body portion 702 may include a first leg 706, a second leg 708, and a third leg 710. The first leg 706 and the third leg 710 may extend generally parallel to one another and generally orthogonal to a plane of the shelf 402c-e. The second leg 708 may extend generally orthogonal to and between the first leg 706 and the third leg 710. In some examples, the first leg 706 may have a height 712 that is less than a height 714 of the third leg 710, although this is not required. In some examples, the first leg 706 and the third leg 710 may have approximately equal heights 712, 714 or the height 712 of the first leg 706 may be greater than the height 714 of the third leg 710.

The first leg 706 may have a width 716 that increases from a first end 718 thereof to an intermediate location 720 between the first end 718 and a second end 722 thereof. The width 716 may increase in a tapered or gradual manner from the first end 718 to the intermediate location 720. At the intermediate location 720, the width 716 may decrease in an abrupt or stair step manner to create a protrusion or shelf 724. The shelf 724 may be configured to rest against an upper surface 434 of the ledge 432 of the shelf 402c-e. In some cases, the first leg 706 may include a first adjustment mechanism 726 configured to adjust length of the first leg 706 to adjust a distance 728 between the shelf 724 and an upper surface of the second leg 708. The first adjustment mechanism 726 may take any form configured to lengthen or shorten the distance 728 between the shelf 724 and the upper surface of the second leg 708. In some examples, the first adjustment mechanism 726 may include a telescoping mechanism with a securing mechanism (e.g., pins and mating apertures, friction fits, or the like), a ratcheted adjustment mechanism, or the like. It is contemplated that the hanger 700 may be assembled with the shelf 402c-e with the first leg 706 in a first configuration. In the first configuration the distance 728 between the shelf 724 and the upper surface of the second leg 708 may be greater than a thickness of the ledge 432. This may allow the first leg 706 of the hanger 700 to be easily positioned over the ledge 432. Once the shelf 724 is positioned over the ledge 432, the first adjustment mechanism 726 may be actuated to reduce the distance 728 to position the shelf 724 against an upper surface 434 of the ledge 432 and the second leg 708 against a lower surface 436 of the ledge 432 such that the ledge 432 is gripped between the shelf 724 and the second leg 708. It is contemplated that in some embodiments, the shelf 724 and/or the second leg 708 may not contact or may only partially contact the ledge 432.

In some cases, the second leg 708 may include a second adjustment mechanism 730 configured to adjust a length 732 of the second leg 708. The length 732 of the second leg 708 may be the distance between a surface 734 of the first leg 706 oriented towards the ledge 432 and a surface 736 of the third leg 710 oriented towards the perimeter edge 428. The second adjustment mechanism 730 may take any form configured to lengthen or shorten the length 732 of the second leg 708. In some examples, the second adjustment mechanism 730 may include a telescoping mechanism with a securing mechanism (e.g., pins and mating apertures, friction fits, or the like), a ratcheted adjustment mechanism, or the like. It is contemplated that the hanger 700 may be assembled with the shelf 402c-e with the second leg 708 in a first configuration. In the first configuration the length 732 of the second leg 708 may be greater than a length and/or width 438 (extending from an inward most point to an outward most point) of the ledge 432. This may allow the first leg 706 of the hanger 700 to be easily positioned over the ledge 432. Once the shelf 724 is positioned over the ledge 432, the second adjustment mechanism 730 may be actuated to reduce the length 732 of the second leg 708 to position the surface 736 of the third leg 710 against the perimeter edge 428 and the surface 734 of the first leg 706 against the ledge 432 such that the ledge 432 is gripped between the first leg 706 and the third leg 710. It is contemplated that in some embodiments, the surface 736 of the third leg 710 and/or the surface 734 of the first leg 706 may not contact or may only partially contact the perimeter edge 428 or the ledge 432. Actuation of the first adjustment mechanism 726 and the second adjustment mechanism 730 may be performed substantially simultaneously or one after the other, as desired. It is further contemplated that actuation of the first adjustment mechanism 726 and/or the second adjustment mechanism 730 may be reversed to length the first leg 706 and/or the second leg 708 to remove the hanger 700.

The hook 704 may be curved member coupled to or formed as a single monolithic structure with the third leg 710. The hook 704 may have a free end 738 spaced a distance from the third leg 710 to define a gap or recess 740 between the curved hook 704 and the third leg 710. The handle 316 of a reservoir 302 may pass through the gap 740 to allow the handle 316 of the reservoir 302 to rest on an inner surface of the hook 704 to support the reservoir 302. While not explicitly shown, the hanger 700 may include one or more features, such as, but not limited to, brackets, hooks, clamps, or the like to receive the tube set when not in use.

While the hanger 700 is shown and described as being coupled with a lower portion of the shelf 402c-e, in some embodiments, the hanger 700 may be inverted and coupled to the raised perimeter 418 of a shelf 402a. It is contemplated that in such an instance, hook 704 may have a “C” shape configured to position the free end 738 of the hook 704 closer to the third leg 710.

In some embodiments, the hanger 700 may be formed as a single monolithic structure. In other embodiments, the hanger 700 may be formed from two or more components that are subsequently coupled together. The hanger 700 may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like. It is further contemplated that the hanger 700 may be formed from a material which is chemically resistant to allow the user to use bacteriostatic and bactericidal cleaners on and around the hanger 700. In some examples, the hanger 700 may be formed, at least in part, with materials having bactericidal properties, such as, but not limited to, ionic-silver, copper or other chemical species or compounds known to inhibit microorganism growth.

The hanger 700 may be positioned at any location along the perimeter of the shelves 402a-e, as desired. As the hanger 700 is configured to clamp to the shelf 402a-e, the hanger 700 may be free from other securement mechanisms. However, the hanger 700 may be fixedly or releasably secured to the shelf 402a-e using additional securement mechanisms. For example, the hanger 700 may be secured to the shelf 402a-e using adhesive, adhesive tape, screws, bolts, snap locks, anchors, hook and loop strips, or the like.

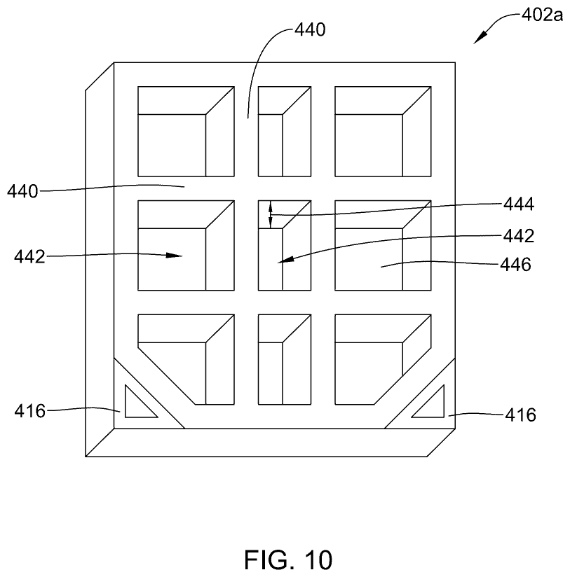

FIG. 9 is a schematic side view of another illustrative hanger 800 that may be used to hang a reservoir 302 relative to the cart 400 with the shelf 402c-e in cross-section. The cart shelf 402c-e may have a generally planar upper surface, although this is not required. The illustrative hanger 800 may be used with shelves having a raised perimeter or upwardly extending lip, as desired. The cart shelf 402c-e may include a downwardly extending (e.g., towards the wheels 406) portion or perimeter edge 428 and an inwardly extending portion 430. The inwardly extending portion 430 may extend towards a center of the shelf 402c-e and may extend generally parallel to the main portion of the shelf 402c-e. The downwardly extending perimeter edge 428 may extend generally orthogonal to and between the main portion of the shelf 402c-e and the inwardly extending portion 430. The inwardly extending portion 430 may have a length and/or width that is less than length and/or width of the shelf 402c-e to define a rim or ledge 432 extending about a perimeter of the shelf 402c-e below a surface 424 of the shelf 402c-e.

The hanger 800 may be configured to clamp to the perimeter edge 428 of the shelf 402a-e. The hanger 800 may include a generally “U” shaped body portion 802 configured to be releasably secured to the shelf 402c-e and a curved hook portion 804 configured to receive the handle 316 of the reservoir 302. The body portion 802 may include a first leg 806, a second leg 808, and a third leg 810. The first leg 806 and the third leg 810 may extend generally parallel to one another and generally orthogonal to a plane of the shelf 402c-e. The second leg 808 may extend generally orthogonal to and between the first leg 806 and the third leg 810. In some examples, the first leg 806 may have a height 812 that is greater than a height 814 of the third leg 810, although this is not required. In some examples, the height of the first leg 806 may be less than or approximately equal to the height 814 of the third leg 810.

The first leg 806 may include a through hole 816 extending through a thickness thereof. The through hole 816 may extend generally parallel to the second leg 808. In some examples, the through hole 816 may include a plurality of internal threads 818 configured to mate with a plurality of external threads 820 on a surface of a clamp mechanism 822. The clamp mechanism 822 may include an elongate shaft 824 having the plurality of external threads 820 along a length thereof and a head portion 826. The head portion 826 may have a greater cross-sectional dimension that the elongate shaft 824. For example, the head portion 826 may be sized and shaped to distribute a load of the reservoir 302 over a sufficient surface area to allow the hanger 800 to support the weight of the reservoir 302. The clamp mechanism 822 may be actuated to axially displace the clamp mechanism 822 along the longitudinal axis thereof. For example, rotation of the clamp mechanism 822 in a first direction may move the head portion 826 towards an inner surface of the perimeter edge 428 and rotation of the clamp mechanism 822 in a second direction, opposite the first direction, may move the head portion 826 away from the inner surface of the perimeter edge 428. The proximal end of the clamp mechanism 822 may include an optional interface 834 to facilitate tightening or loosening the clamp mechanism. For example, the interface 834 may tighten or loosen how much the head portion 826 engages with the perimeter edge 428. An illustrative interface 834 may include but is not limited to a fixed T-nut, a fixed wing nut, a thumb screw, or the like. The interface 834 may be positioned on either side of the first leg 806, as desired. While the clamp mechanism 822 is shown and described as having a threaded engagement with the first leg 806, the clamp mechanism 822 may be movably secured to the first leg 806 using other mechanisms, as desired. In some cases, the clamp mechanism 822 may be movably secured to the first leg 806 using a ratcheted mechanism.

It is contemplated that the hanger 800 may be assembled with the shelf 402c-e with the clamp mechanism 822 in a first configuration. In the first configuration, the head portion 826 may be positioned against or adjacent to the first leg 806. This may allow the body portion 802 to be positioned with the first leg 806 inward to the perimeter edge 428, the second leg 708 adjacent to the ledge 432, and the third leg 810 adjacent to an outer surface of the perimeter edge 428. Once the body portion 802 is in the desired position, the clamp mechanism 822 may be actuated to bring an end surface 828 of the head portion 826 in contact with the inner surface of the perimeter edge 428 and to bring a surface of the third leg 810 into contact with outer surface of the perimeter edge 428 such that the perimeter edge 428 is gripped between the head portion 826 of the clamp mechanism 822 and the third leg 810. It is contemplated that in some embodiments, the third leg 810 may not contact or may only partially contact the perimeter edge 428.

The hook 804 may be curved member coupled to or formed as a single monolithic structure with the third leg 810. The hook 804 may have a free end 830 spaced a distance from the third leg 810 to define a gap or recess 832 between the curved hook 804 and the third leg 810. The handle 316 of a reservoir 302 may pass through the gap 832 to allow the handle 316 of the reservoir 302 to rest on an inner surface of the hook 804 to support the reservoir 302. While not explicitly shown, the hanger 800 may include one or more features, such as, but not limited to, brackets, hooks, clamps, or the like to receive the tube set when not in use.

While the hanger 800 is shown and described as being coupled with a lower portion of the shelf 402c-e, in some embodiments, the hanger 800 may be inverted and coupled to the raised perimeter 418 of a shelf 402a. It is contemplated that in such an instance, hook 804 may have a “C” shape configured to position the free end 830 of the hook 804 closer to the third leg 810.

In some embodiments, the hanger 800 may be formed as a single monolithic structure. In other embodiments, the hanger 800 may be formed from two or more components that are subsequently coupled together. The hanger 800 may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like. It is further contemplated that the hanger 800 may be formed from a material which is chemically resistant to allow the user to use bacteriostatic and bactericidal cleaners on and around the hanger 800. In some examples, the hanger 800 may be formed, at least in part, with materials having bactericidal properties, such as, but not limited to, ionic-silver, copper or other chemical species or compounds known to inhibit microorganism growth.

The hanger 800 may be positioned at any location along the perimeter of the shelves 402a-e, as desired. As the hanger 800 is configured to clamp to the shelf 402a-e, the hanger 800 may be free from other securement mechanisms. However, the hanger 800 may be fixedly or releasably secured to the shelf 402a-e using additional securement mechanisms. For example, the hanger 800 may be secured to the shelf 402a-e using adhesive, adhesive tape, screws, bolts, snap locks, anchors, hook and loop strips, or the like.

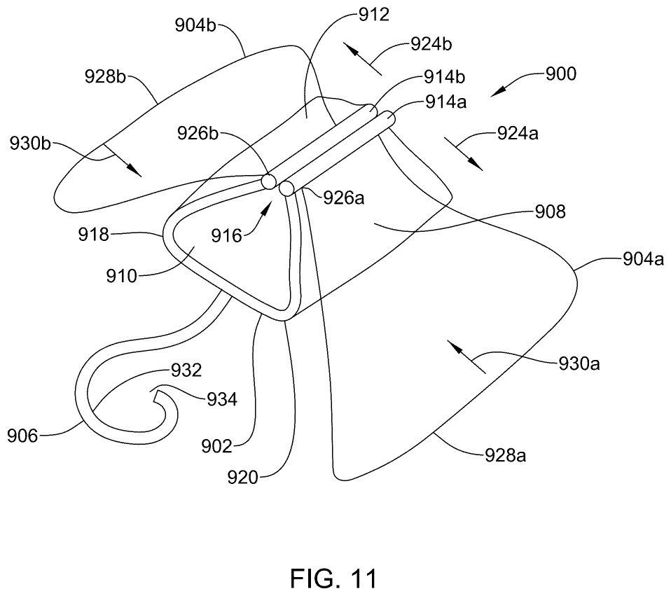

In some embodiments, it may be desirable to hang or otherwise secure the reservoir 302 relative to an underside of a cart shelf 402a-e. FIG. 10 is a bottom perspective view of an illustrative cart shelf 402a. While the bottom is described within respect to the top shelf 402a, any of the shelves 402b-e may have a similar underside structure. The underside of the cart shelf 402a may include a plurality of intersecting ribs 440 defining a plurality of open cells 442. The ribs 440 may be in one or more rows and one or columns with each row and column laterally spaced from the adjacent row or column. The rows may be generally parallel to one another, and the columns may be generally parallel to one another. In some cases, the rows may extend generally orthogonal to the columns such that the cells have a generally rectangular cross-sectional shape. However, the ribs 440 may be arranged in non-orthogonal and/or non-parallel configurations to form cells 442 of different shapes. The cells 442 may have depth 444 extending from a lowermost surface of the ribs 440 to a lower surface of the shelf 402a. The sidewalls of the cells 442 may be defined by the walls of the ribs 440.

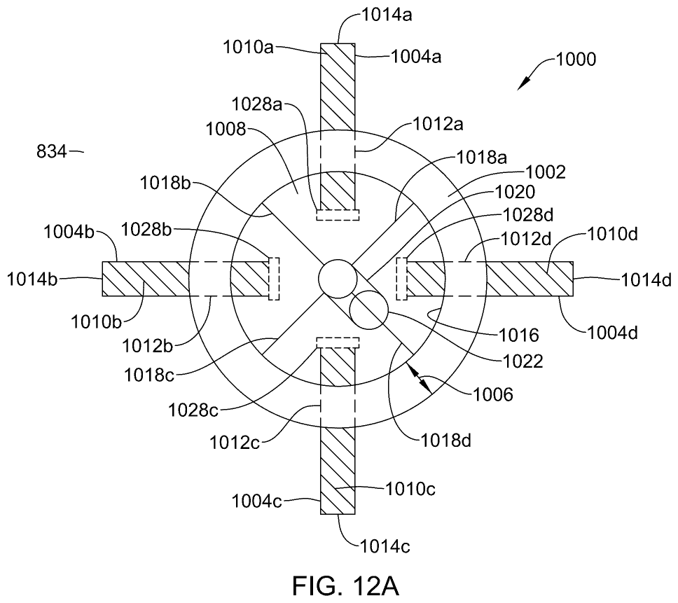

FIG. 11 is a perspective view of another illustrative hanger 900 that may be used to hang a reservoir 302 relative to the cart 400. The hanger 900 may generally include a body portion 902, a first actuation mechanism 904a and a second actuation mechanism 904b, and a hook 906. The body portion 902 may be a single monolithic strip of material bent into a generally triangular prism having a first side 908, a second side 910, and a third side 912. The body portion 902 may have a first free end 914a and a second free end 914b. The free ends 914a-b may movably meet at a first apex 916 of the triangular prism. The body portion 902 may be configured to flex or bend at the second apex 920 and the third apex 922 thereof in response to an applied force and return to an unbiased configuration (as shown in FIG. 11) in the absence of an applied force. It is contemplated that the body portion 902 may be formed from a material configured to return to an original shape, such as, but not limited to, spring steel. More particularly, in response to an applied force, the free ends 914a-b of the body portion 902 may move away from one another as shown at arrows 924a, 924b.

The first actuation mechanism 904a may be coupled at a first end 926a thereof to the first end 914a of the body portion 902 and the second actuation mechanism 904b may be coupled at a first end 926b thereof to the second end 914b of the body portion 902. The first and second actuation mechanisms 904a, 904b may extend from a respective first end 926a, 926b to a respective second end 928a, 928b. In some embodiments, the first and second actuation mechanisms 904a-b may be formed from a bent elongate wire where both free ends of the wire are coupled to the free ends 914a-b of the body portion 902. However, this is not required. In other examples, the first and second actuation mechanisms 904a-b may be generally planar elements. The second ends 928a-b of the first and second actuation mechanisms 904a-b may be biased towards one another, as shown at arrows 930a, 930b, to move the free ends 914a-b of the body portion 902 away from one another.

When the free ends 914a-b of the body portion 902 are moved away from one another the sides 908, 910, 912 of the body portion 902 may form a generally “U” shaped configuration defining an open channel to allow the body portion 902 to be positioned over a rib 440. The hanger 900 may be positioned over one of the ribs 440 on the underside of the shelf 402a-e such that the first side 908 of the body portion 902 is disposed on a first side of a rib 440 and the third side 912 of the body portion 902 is disposed on a second side of the rib 440 while the second side 910 spans a width of the rib 440. When the biasing force is removed from the second ends 928a-b of the first and second actuation mechanisms 904a-b, the free ends 914a-b of the body portion 902 attempt to move towards one another. Said differently, the free ends 914a-b of the body portion 902 may be biased to return to the unbiased generally triangular prism shape. When disposed over the rib 440, the free ends 914a-b are precluded from completely returning to the unbiased configuration such that a compressive force is exerted on the rib 440 to clamp the hanger 900 to the rib 440. In some examples, the free ends 914a-b may include teeth or serrations configured to penetrate the surface of the rib 440 to further secure the hanger 900 to the rib 440.

The hook 906 may extend from the second side 910 of the body portion 902 in a direction generally opposite the first apex 916 thereof. The hook 906 may include a curved segment 932 configured to receive the handle 316 of the reservoir 302. The curved segment 932 may extend laterally from a central axis thereof before curving back on itself and extending laterally outwards and upwards. The curved segment 932 may form less than a 360° arc. For example, the curved segment 932 may include an opening 934 for allowing the handle 316 of a reservoir 302 to pass therethrough. The handle 316 of the reservoir 302 may rest on an inner surface of the curved segment 932 to support the reservoir 302. While not explicitly shown, the hanger 900 may include one or more features, such as, but not limited to, brackets, hooks, clamps, or the like to receive the tube set when not in use.

In some embodiments, the hanger 900 may be formed as a single monolithic structure. In other embodiments, the hanger 900 may be formed from two or more components that are subsequently coupled together. The hanger 900 may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like. It is further contemplated that the hanger 900 may be formed from a material which is chemically resistant to allow the user to use bacteriostatic and bactericidal cleaners on and around the hanger 900. In some examples, the hanger 900 may be formed, at least in part, with materials having bactericidal properties, such as, but not limited to, ionic-silver, copper or other chemical species or compounds known to inhibit microorganism growth.

The hanger 900 may be positioned at any location along the perimeter of the shelves 402a-e and/or to any of the underside ribs 440 thereof, as desired. As the hanger 900 is configured to clamp to the shelf 402a-e, the hanger 900 may be free from other securement mechanisms. However, the hanger 900 may be fixedly or releasably secured to the shelf 402a-e using additional securement mechanisms. For example, the hanger 900 may be secured to the shelf 402a-e using adhesive, adhesive tape, screws, bolts, snap locks, anchors, hook and loop strips, or the like.

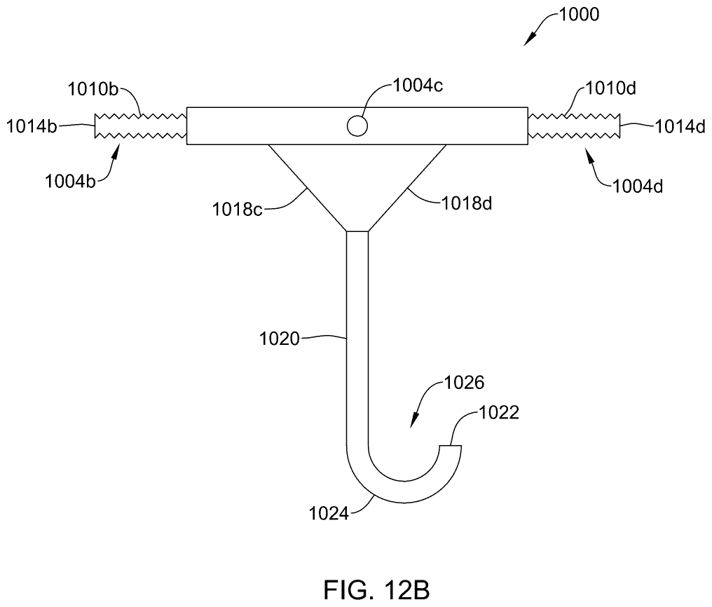

FIG. 12A is a top view of another illustrative hanger 1000 that may be used to hang a reservoir 302 relative to the cart 400. FIG. 12B is a side view of the illustrative hanger 1000 of FIG. 12A. Generally, the hanger 1000 may be configured to be secured within a cell 442 of the underside of the shelf 402a-e. The hanger 1000 may include a body portion 1002 and a plurality of coupling mechanisms 1004a-d. The body portion 1002 may be sized and shaped to fit within one of the cells 442 of the shelf 402a-e. For example, the body portion 1002 may have a height, width and/or length less than a height, width, and/or length of the cell 442. In some cases, the body portion 1002 may have a generally annular shape having a wall thickness 1006 and defining a central opening 1008. In other examples, the body portion 1002 may have a non-circular outer and/or inner shape.

While the illustrated embodiment includes four coupling mechanisms 1004a-d, the hanger 1000 may include fewer than four or more than four coupling mechanisms 1004a-d, as desired. In some examples, the coupling mechanisms 1004a-d may each have a plurality of external threads 1010a-d. For example, the coupling mechanisms 1004a-d may be threaded shafts, threaded set screws, or the like. The coupling mechanisms 1004a-d may be configured to extend through mating apertures or through holes 1012a-d extending through the wall thickness 1006 of the body portion 1002. While the illustrated body portion 1002 includes four through holes 1012, the body portion 1002 may include fewer than four or more than four through holes 1012, as desired. It is contemplated that the hanger 1000 may include a same number of through holes 1012a-d and coupling mechanisms 1004a-d. The external threads 1010a-d of the coupling mechanisms 1004a-d may be configured to threadably engage mating threads formed in the through holes 1012a-d. Rotation of the coupling mechanisms 1004a-d in a first direction may move a radially outward edge 1014a-d in a radially outward direction. Rotation of the coupling mechanisms 1004a-d in a second direction opposite the first may move the radially outward edge 1014a-d in a radially inward direction. The radially outward edge 1014a-d of the coupling mechanisms 1004a-d may each include an optional interface 1028a-d to facilitate tightening or loosening the coupling mechanisms 1004a-d. For example, the interface 1028a-d may tighten or loosen how much the radially outward edge 1014a-d engages with walls of the cell 442. An illustrative interface 1028a-d may include but is not limited to a fixed T-nut, a fixed wing nut, a thumb screw, or the like. The interface 1028a-d may be positioned on either side of the body portion 1002, as desired.

The hanger 1000 may further include a hook 1020. The hook 1020 may be secured to the body portion 1002 by one or more fastening members 1018a-d. The fastening members 1018a-d may extend from a radially inner surface 1016 of the body portion 1002 to the hook 1020. The hook 1020 may be configured to hang from a central region of the opening 1008, although this is not required. In some cases, the hook 1020 may be positioned closer to the radially inner surface 1016, if so desired. While the illustrated embodiment includes four fastening members 1018a-d, the hanger 1000 may include fewer than four or more than four fastening members 1018a-d, as desired. The fastening members 1018a-d may be circumferentially offset from the coupling mechanisms 1004a-d to allow for free movement of the coupling mechanisms 1004a-d, although this is not required.

The hook 1020 may include a curved segment 1024 configured to receive the handle 316 of the reservoir 302. The curved segment 1024 may form less than a 360° arc. For example, the curved segment 1024 may include an opening 1026 for allowing the handle 316 of a reservoir 302 to pass therethrough. The handle 316 of the reservoir 302 may rest on an inner surface of the curved segment 1024 to support the reservoir 302. While not explicitly shown, the hanger 1000 may include one or more features, such as, but not limited to, brackets, hooks, clamps, or the like to receive the tube set when not in use.

To assemble the hanger 1000 with the shelf 402a-e, the coupling mechanisms 1004a-d may be actuated such that the radially outward edges 1014a-d are in a radially inward position or radially contracted configuration. It is contemplated that the coupling mechanisms 1004a-d need not be in a most radially inward position to couple the hanger 1000 with the shelf 402a-e. For example, the coupling mechanisms 1004a-d may be positioned at a point between the most radially inward position and the most radially outward position which gives the hanger 1000 a maximum outer dimension less than the width and/or length of the cell 442 into which it is to be inserted. The body portion 1002 may then be inserted into a cell 442 of the shelf 402a-e. The coupling mechanisms 1004a-d may then be rotated or otherwise actuated to move the radially outward edge 1014a-d radially outwards. The coupling mechanisms 1004a-d may be rotated until the radially outward edge 1014a-d of each coupling mechanism 1004a-d engages a surface of the cell 442. In some cases, the radially outward edge 1014a-d of each coupling mechanism 1004a-d may engage a flat wall of the cell 442 (e.g., the ribs 440). In other embodiments, the radially outward edge 1014a-d of each coupling mechanism 1004a-d may engage a corner of the cell 442 (e.g., an intersection point of two or more ribs 440). The coupling mechanisms 1004a-d may exert a radially outward force on the walls of the cells 442 to secure the hanger 1000 within the cell 442. When the hanger 1000 is secure relative to the cell 442, the hook 1020 may be positioned below the shelf 402a-e to receive the reservoir 302.

In some embodiments, the hanger 1000 may be formed as a single monolithic structure. In other embodiments, the hanger 1000 may be formed from two or more components that are subsequently coupled together. The hanger 1000 may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like. It is further contemplated that the hanger 1000 may be formed from a material which is chemically resistant to allow the user to use bacteriostatic and bactericidal cleaners on and around the hanger 1000. In some examples, the hanger 1000 may be formed, at least in part, with materials having bactericidal properties, such as, but not limited to, ionic-silver, copper or other chemical species or compounds known to inhibit microorganism growth.

The hanger 1000 may be positioned within any cell 442 of the underside of the shelf 402a-e, as desired. As the hanger 1000 is configured to couple to the shelf 402a-e, the hanger 1000 may be free from other securement mechanisms. However, the hanger 1000 may be fixedly or releasably secured to the shelf 402a-e using additional securement mechanisms. For example, the hanger 1000 may be secured to the shelf 402a-e using adhesive, adhesive tape, screws, bolts, snap locks, anchors, hook and loop strips, or the like.

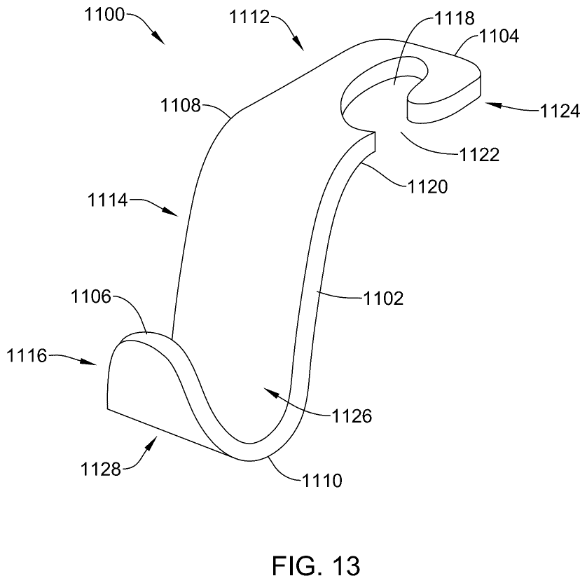

FIG. 13 is a perspective view of another illustrative hanger 1100 that that may be used to hang a reservoir 302 relative to the cart 400. The hanger 1100 may include a body portion 1102 extending from a first end 1104 to a second end 1106. The body portion 1102 may include a first region 1112 extending from the first end 1104 to a first curved region 1108, a second region 1114 extending from the first curved region 1108 to a second curved region 1110, and a third region 1116 extending from the second curved region 1110 to the second end 1106. The first curved region 1108 and the second curved region 1110 may have different angles of curvature. In some cases, the first curved region 1108 may have a curve or bend in the range of about 75° to about 115° and the second curved region 1110 may have a curve or bend in the range of about 165° to about 195°. These are just some examples. It is contemplated that the first and/or second curved regions 1108, 1110 may have other ranges of curvature, as desired.

The body portion 1102 may further include an aperture 1118 extending through a thickness thereof. In some cases, the aperture 1118 may extend to a lateral side 1120 of the body portion 1102 to define an opening 1122 from the lateral side 1120 to the aperture 1118. The aperture 1118 and the opening 1122 may be sized and shaped to form a first hook region 1124 adjacent the first end 1104 of the body portion 1102. It is contemplated that the first hook region 1124 may be used to hook the hanger 1100 to various parts of the cart 400, such as, but not limited to, poles 410, handles 416, or the like. The first hook region 1124 may be configured to secure the hanger 1100 to features of the cart 400 which extend parallel to a surface 424 of the shelves 402a-e or to features of the cart 400 which extend at non-parallel angles to the surface of the shelves 402a-e.

A portion of the second region 1114, the second curved region 1110, and the third region 1116 may form a “U” shaped structure defining a channel 1126. The “U” shaped structure may define a second hook region 1128. It is contemplated that the second hook region 1128 may be used to hook the hanger 1100 to various parts of the cart 400, such as, but not limited to, the handles 416, perimeter edges, or the like. The second hook region 1128 may be configured to secure the hanger 1100 to features of the cart 400 which extend parallel to a surface 424 of the shelves 402a-e.

When the first hook region 1124 is hung from or coupled to the cart 400, the reservoir 302 may be hung from the second hook region 1128. When the second hook region 1128 is hung from or coupled to the cart 400, the reservoir may be hung from the first hook region 1124. It is contemplated that having a hanger 1100 with differing hook styles may provide some flexibility in where the hanger 1100 is hung from the cart 400.

In some embodiments, the hanger 1100 may be formed as a single monolithic structure. In other embodiments, the hanger 1100 may be formed from two or more components that are subsequently coupled together. The hanger 1100 may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like. In some examples, the hanger 1100 may be formed from a flexible material allowing the hanger 1100 to be at least temporarily deformed to hang from the cart 400. In other embodiments, the hanger 1100 may be formed from a more rigid material that forms a snap fit or friction fit with the cart 400. However, a tight fit between the hanger 1100 and the cart 400 is not required. In yet other embodiments, it is further contemplated that the hanger 1100 may be formed from a material which is chemically resistant to allow the user to use bacteriostatic and bactericidal cleaners on and around the hanger 1100. In some examples, the hanger 1100 may be formed, at least in part, with materials having bactericidal properties, such as, but not limited to, ionic-silver, copper or other chemical species or compounds known to inhibit microorganism growth.

The hanger 1100 may be positioned at any location along the perimeter of the shelves 402a-e and/or to any of the underside ribs 440 thereof, as desired. As the hanger 1100 is configured to clamp to the shelf 402a-e, the hanger 1100 may be free from other securement mechanisms. However, the hanger 1100 may be fixedly or releasably secured to the shelf 402a-e using additional securement mechanisms. For example, the hanger 1100 may be secured to the shelf 402a-e using adhesive, adhesive tape, screws, bolts, snap locks, anchors, hook and loop strips, or the like.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A hanger for hanging a fluid reservoir relative to an endoscope cart, the hanger comprising:
a generally planar base plate;
an arched member extending from an upper surface of the base plate, the arched member including a first generally “U” shaped region and a second generally “U” shaped region; and
at least one arm extending from the arched member, the at least one arm configured to receive a tubing;
wherein the second generally “U” shaped region is configured to receive a portion of a fluid reservoir.

2. The hanger of claim 1, wherein the first generally “U” shaped region extends from the upper surface of the base plate.

3. The hanger of claim 1, wherein the first generally “U” shaped region has a height greater than a raised perimeter of a shelf of an endoscope cart.

4. The hanger of claim 1, wherein the second generally “U” shaped region curves away from a lateral edge of the base plate.

5. The hanger of claim 1, wherein the base plate has a generally pentagonal shape.

6. The hanger of claim 5, wherein an apex of the base plate is rounded.

7. The hanger of claim 1, further comprising a gasket secured to a lower surface of the base plate.

8. The hanger of claim 1, wherein the at least one arm extends generally parallel to a plane of the base plate.

9. The hanger of claim 1, further comprising a clamp at a free end of the at least one arm.

10. The hanger of claim 1, wherein the hanger is formed at least in part, from ionic silver or copper.

11. The hanger of claim 1, wherein the hanger is a single monolithic structure.

12. A hanger for hanging a fluid reservoir relative to an endoscope cart, the hanger comprising:
a body portion defining a central opening;
a plurality of coupling mechanisms extending through a wall of the body portion, the plurality of coupling mechanisms movable between a radially extended and a radially contracted configuration; and
a hook secured to the body portion.

13. The hanger of claim 12, wherein the hook is secured to the body portion by one or more fastening members.

14. The hanger of claim 13, wherein the one or more fastening members are circumferentially offset from the plurality of coupling mechanisms.

15. The hanger of claim 12, wherein when in the radially extended configuration, the plurality of coupling mechanisms are configured to engage one or more ribs of a shelf of a cart.

16. A hanger for hanging a fluid reservoir relative to an endoscope cart, the hanger comprising:
a generally “U” shaped body portion having a first leg including first adjustment mechanism, a second leg including a second adjustment mechanism, and a third leg, the second leg extending generally orthogonal to and between the first leg and the third leg; and
a curved hook portion extending from the third leg;
wherein a portion of the first leg is configured to engage a ledge of a shelf of an endoscope cart.

17. The hanger of claim 16, wherein the first adjustment mechanism is configured to adjust a length of the first leg.

18. The hanger of claim 16, wherein the second adjustment mechanism is configured to adjust a length of the second leg.

19. The hanger of claim 16, wherein a width of the first leg increases from a first end to an intermediate location and the width of the first leg decreases in a stair-step manner at the intermediate location.

20. The hanger of claim 16, wherein the first or second adjustment mechanism comprises a ratcheted adjustment mechanism.
